(19) United States
(12) Patent Application Publication
(10) Pub. No.: US 2021/0259858 A1
(43) Pub. Date: Aug. 26, 2021

(54) TRAINING DEVICE FOR STIMULATING NERVE CELL ENDS IN A TRAINING MANNER, AND A CORRESPONDING PROSTHESIS

(71) Applicant: Saphenus Medical Technology GmbH, Krems an der Donau (AT)

(72) Inventors: Bernhard Zach, Wien (AT); Martin Brandstätter, Wien (AT); Aaron Pitschl, Wien (AT); Rainer Schultheis, Wien (AT); Andreas Mühlenberend, Weimar (DE)

(73) Assignee: Saphenus Medical Technology GmbH, Krems an der Donau (AT)

(21) Appl. No.: 17/057,270

(22) PCT Filed: May 21, 2019
(86) PCT No.: PCT/IB2019/054197

§ 371 Date: Nov. 20, 2020

(30) Foreign Application Priority Data
May 30, 2018 (EP) 18175203.1

(51) Int. Cl. A61F 2/68; G06N 20/00; G16H 40/63; A61N 1/36; A61F 2/78; A61F 2/66

(52) U.S. Cl. CPC A61F 2/68; G06N 20/00; G16H 40/63; A61N 1/3603; A61F 2002/7818; A61F 2/66; A61F 2002/6827; A61F 2002/665; A61F 2/7812

(57) ABSTRACT

A training device for stimulating nerve cell ends in a training manner, includes a housing having at least one first housing portion and a plurality of stimulators for stimulating physionomie-typical nerve area portions. A plurality of stimulators are arranged systematically at least on the first housing portion of the housing so that the plurality of stimulators in the training state each act on nerve cell ends with which they are associated. Also disclose are a method for operating the training device, the use of the training device as a modular stimulation device for stimulating nerve cell ends, a prosthesis, a method for producing a prosthesis and the use of a training device for stimulating nerve cell ends in a prosthesis.

The invention relates to a training device for stimulating nerve cell ends in a training manner, a prosthesis, as well as a method for operating a training device, and a method for producing a prosthesis, as well as the use of a training device for stimulating nerve cell ends, and the use of a training device in a prosthesis according to the generic terms of the independent claims.

On Jun. 8, 2015, the daily newspaper derStandard.at, under the rubric Science (Wissenschaft)>Human Being (Mensch), published an article that provides information on the development by Professor Hubert Egger, one of the world's leading prosthesis researchers. There, the first leg prosthesis is reported, which “feels along with”. It is explained in detail that, in the case of an amputated patient, severed nerves were reactivated and relocated into to a skin area on the leg stump. Since this skin area subsequently comprises reinnervated nerve cell ends, this skin area becomes particularly sensitive. The nerve endings were relocated in such a way that the former foot was depicted in this skin area with its physionomy-typical nerve area portions by the nerve ends relocated there. In other words, a physionomy-typical nerve area portion is a natural portion of the nerve area, which is relocated to the surface of a skin area that is actually untypical for this purpose. Therefore, the patient did not (only) feel the skin (surface) at this skin area as usual, but the reinnervated nerve cell ends and thus virtually his/her foot sole, which is no longer present due to amputation of the extremity.

The prosthesis used in the presented solution was designed in such a way that the stimulators are arranged fixed in the shaft of the prosthesis in such a way that a stimulation of the nerve cell ends is only possible after the individual orthoptic adaptation of the prosthesis shaft in the patient.

US 2014/0277583 A1 discloses a system for a neural-activatable limb prosthesis. The system comprises an instrumented prosthesis with sensors, which are connected by means of an interface system to a stimulation system for digitizing and associating the sensor values. The interface system comprises a stimulator, which interacts with an electrode arranged within the limb.

The disadvantage of this known system is that an electrode arranged within the limb is necessary to stimulate a nerve in the limb. Furthermore, this system necessarily includes a prosthesis to transfer sensor values from the prosthesis to a nerve in such a way that this system is not suitable as a training device as described here in the present case.

US 2008/0077192 A1 discloses a device and a method for neuro-stimulation. The device comprises stimulation electrodes to stimulate nerve cells that are naturally present. The stimulations are transferred from the stimulation electrodes to a contact surface of the device housing. This stimulates the surface of the body on a large scale.

The disadvantage of this known solution is that the stimulation electrodes do not touch a patient's body and are therefore not designed to stimulate individual nerve cell ends associated with them.

US 2003/0144710 A1 shows a device similar to this type. None of the aforementioned devices shows a training device for the stimulation of nerve cell ends in a training manner.

It is the object of the present invention to remedy one or a plurality of disadvantages of prior art. A well-known phenomenon is that reinnervated nerve cell ends—since they come to rest in an area that is alien to them, thereby taking relatively long to be fully responsive. In particular, therefore, a training device for stimulating of nerve cell ends in a training manner should be created, with which it is postoperatively possible, in a timely manner, a natural movement of a missing extremity at the nerve cell ends of the patient in an ultrasensitive manner. This is intended to help to establish the full functionality of the affected nerve cell ends as quickly as possible, in particular, by means of nerve cell growth (cell division at the synapses). Furthermore, a method for operating a training device as well as a prosthesis for the use of this training device, a method for producing this prosthesis, and the use of the training device for stimulation in a training manner, as well as the use of the training device in a prosthesis for another purpose are provided. This is intended to remedy one or a plurality of disadvantages of prior art and to be capable of better ensuring what is strived after for the intended purpose, namely the stimulation of growth or improvement of the sensitivity of the nerve cell ends.

This task is achieved by means of the features of the independent claims. Favourable further embodiments are presented in the figures, the corresponding description and in the dependent patent claims.

The training device according to the invention for stimulating of nerve cell ends in a training manner comprises a housing with at least one first housing portion, as well as with a plurality of stimulators for stimulating physionomy-typical nerve area portions. A plurality of stimulators are systematically arranged on the first housing portion of the housing so that the plurality of stimulators in the training state can act on the respectively associated nerve cell ends.

The systematics of the arrangement depend on the actual position of the respectively relevant nerve cell endings on the physionomy-typical postoperatively provided nerve area portion so that, in the case of the patient, a sequential response of the respective stimulator of the respective stimulators from the plurality of stimulators generates the feeling of a real stimulation along the actual or natural skin area portion of the missing extremity and is perceived in the brain as such. When the plurality of stimulators act on the nerve cell ends associated with them, stimulation signals are transmitted from the stimulators to the nerve cell ends. This allows the nerve cell ends to be trained as early on as possible so that the acclimatization phase of the patient for a prosthesis subsequently following is minimized in terms of time.

A transfer of information or information transmission is also always understood as stimulating, wherein, typically, no energy transfer from the training device to the patient takes place.

With the training device according to the invention, the feeling of walking with his/her (former) foot for example can be simulated immediately after an operation for the sake of training. Thanks to this training device, it is possible to postoperatively stimulate the growth of the physionomy-typical nerve area portions or nerve cell ends, thereby resulting in a timely and optimal provision of a suitable skin area on the remaining part of the extremity of the patient. In other words, the training device is therefore used to more successfully reinnervate nerve cell ends for the purpose of later adaptation of a remaining part of an extremity to a prosthesis.

In particular, with the training device, reinnervated nerve cell ends can be stimulated, wherein the reinnervated nerve cell ends of a foot can be trained particularly well.

Preferably, the systematic arrangement of the plurality of stimulators depicts a depiction of the gait line of a human foot sole. The plurality of stimulators are arranged according to a matrix, for example, in a lighting-shaped manner. For example, at least five stimulators are required in the first housing portion, whereby the depiction of the gait line of the human foot can be depicted particularly well. The systematic arrangement of the plurality of stimulators is carried out in such a way that each stimulator is arranged at least on a physionomy-typical nerve cell end associated with it, which, in the case of an intact foot, is or would be connected to the respective nerve cell end along the natural gait line of the foot.

Preferably, a plurality of stimulators, in particular, two stimulators, are arranged in the region of each physionomy-typical nerve cell end, whereby the stimulation resolution is improved.

Favourably, the plurality of stimulators are arranged on the training device in an individually configurable manner. The spatial position of a first stimulant can be arranged relative to at least one of the other stimulators. The stimulators can be arranged along a first longitudinal axis on the first housing portion in an adjustable manner. In addition or as an alternative, the stimulators can be rotatably arranged around a fastening portion in a configurable manner. Thus, at least single simulators from the plurality of stimulators can be individually configured to individual nerve cell ends, whereby the training of the nerve cell ends can be further improved.

In particular, according to a special embodiment of the invention, these two stimulators can be designed differently so that, at the respective point, different stimulations or stimulation signals are transferred to the extremity or to the foot. For example, the signal of the first stimulator can signal the virtuality of touch while the signal of the further stimulator is switched on only after a certain simulated pressure intensity of touch has been achieved, thereby being able to render both qualitative as well as quantitative information.

The natural gait line of a foot is usually determined in a gait-line analysis at a measuring station that is suitable for this purpose. The kinematics and kinetics of the gait are measured and displayed along a line on the sole of the foot. Typically, a natural gait line largely runs along the longitudinal extension of the foot sole.

Depending on the individual anatomy of the foot, each foot can comprise a plurality of individual natural gait lines, which are often defined as a single natural gait line. For example, the natural gait lines of a healthy foot differ from the gait lines of an artificial foot on the same patient in terms of their gait-line width, or the single gait line of a wearer of a transfemoral prosthesis differs from the single gait line of a wearer of a transtibial prosthesis. Thus, with the training device described here, gait lines or only one gait line that have been tuned to the patient can be trained on an individual basis.

Favourably, at least in portions, the stimulators comprise a stimulation insulation from an elastic material, such as a rubber material. This stimulation isolation prevents the stimulators from interacting with the housing portion in a sound-transmitting manner. Thus, an additional acoustic decoupling with regard to housing of the training device can be implemented.

Thereby, it depends on where the artificial foot sole has the gait line when used as intended. The plurality of stimulators on the training device become a depiction of the gait line by being arranged at the points that correspond to the corresponding nerve area end portions. The reinnervated nerve cell ends on the physionomy-typical nerve area portion can also be arranged according to a depiction of the gait line, wherein this is not necessarily required. The only thing that is decisive is the correct association of stimulators and nerve cell ends.

Preferably, a control device for controlling the plurality of stimulators is available, which comprises at least one training program. Thus, the plurality of stimulators connected to the control device are stimulated according to a predefined program sequence so that the nerve cell ends can be specifically trained individually as well as collectively, or they can be trained in a reproducible manner.

Preferably, the control device is coupled to a visualization device in order to visually display to the patient a stimulation in a training manner from the at least one training program. During the training of the nerve cell ends, not only the tactile sensors in the region of the physionomy-typical nerve area portion are stimulated, but also the visual receptors in the human eye are stimulated, thereby improving overall perception and processing in the human brain. The training can be supported according to one of the considerations underlying the invention by providing the brain with the same information via other sensory channels. For example, by means of a visual depiction at which point (of the “pictured” foot) the respective training stimulus is emitted.

In addition or as an alternative, messages, such as instructions from a third party, for example, a doctor, can be displayed for the patient on the visualization device.

By a single training program, here, a training program is understood that comprises training data or training commands for certain movement sequences, such as training data on walking movements, bouncing movements or climbing movements for example. Alternatively, for example, —in the case of the reinnervation of finger nerves for a human hand—a gripping movement can be carried out as a training program using the control device.

Preferably, the control device is arranged in the housing, whereby a compact construction of the training device is possible.

Preferably, the control device is controllable with an external remote device, such as a remote control or a computer. The remote device can be connected to the interface of the training device using a data cable. Thus, the training device, which is located at a region of the body that is difficult to access, can be controlled by the user.

In particular, the control device can be controlled with a mobile terminal device, such as a smartphone or a tablet for example. For this purpose, the mobile terminal device has an application, which can be connected to the training device for exchanging data. This means that no additional device is necessary, and it is possible to control the training device with a terminal device that is known or familiar to the user.

Preferably, the training device comprises a data transmission device for transferring data. For example, the user can transmit messages, as data, to the training device, which can be displayed on the visualization device.

Preferably, the data transmission device is arranged in the control device so that external training data can be transferred to the control device. This allows any authorized user to adapt existing training data to the training device or to transfer new training programs to the training device.

By a user, both a patient as well as a physiotherapist, and/or an orthopaedic surgeon are understood here.

In addition or as an alternative, stimulation data are transferred to the control device with the data transmission device if necessary. Stimulation data or stimulation commands are those data that can individually stimulate individual reinnervated nerve cell ends. This allows different sensitive nerve cell ends in the physionomy-typical nerve area portion to be trained to an equally high sensitivity level.

Preferably, the data transmission device comprises a transmitting unit and a receiving unit for wireless data exchange. Typically, the data transmission device comprises a WLAN unit or a Bluetooth® connection, whereby the training data and stimulation data can be easily transferred to the control device.

Preferably, the data transmission device includes an interface on which a user can define training programs. Typically, an input device, such as a keyboard for example, can be connected to the interface so that the training programs can be easily adapted.

In addition or as an alternative, a user can define training programs and stimulation programs on the interface. A stimulation program comprises stimulation commands or stimulation data for stimulating individual nerve cell ends, which can thus be individually adaptable. For example, training programs and stimulation programs can be adapted synchronously.

In particular, the interface includes an input device for entering stimulation data, training data or sensor data. In addition to a main switch (on and off switch), a selection switch for selecting specified training programs and/or stimulation programs is provided as an input device. This facilitates the activation of the training device or the selection of the training programs and/or the stimulation programs for the user.

Preferably, the input device is arranged on the visualization device, whereby the haptics of the training device for the user are improved.

In particular, the input device is integrated in the visualization device as a touchscreen. This makes it possible to produce a particularly lightweight training device.

Preferably, the data transmission device can be powered using external sensor data of a prosthesis. Thereby, external sensor data is transmitted to the data transmission devices and can be used in the associated control device for controlling the plurality of stimulators. For example, external sensor data, such as pressure sensor data, can be sent from a prosthesis part, preferably from an artificial foot sole, to the data transmission device, and can be transmitted to the plurality of stimulators.

In addition or as an alternative, the data transmission device is connected to a plurality of external sensors. In addition to the sensor data from a prosthesis, further data from external sensors, such as a temperature sensor, humidity sensor, a GPS sensor, etc., can also be supplied, which can be used in combination with the sensor data of the prosthesis to train the nerve cell ends. For example, GPS data can be used to record the location of the prosthesis wearer and combine it with a training program in the training device. This allows the training program “mountain climbing”, or “walking”, but also “dancing” to be combined with the current or desired location(s) of the prosthesis wearer.

Preferably, the data transmission device is connected to a cloud in a network for exchanging data, wherein the data can be external sensor data, external training data or external stimulation data. This allows users to access historical data located in the cloud. With the use of the historical data, maintenance work or service applications in the training device can be shortened or improved for example.

Preferably, the data transmission device is connected to an online service for exchanging data, which makes it easy to assign the access rights of the users.

Preferably, the data is stored using blockchain technology, whereby the data is stored in a particularly well-encrypted manner so that no sensitive or personal data of the user can be published.

Preferably, the control device comprises a computing unit, wherein the computing unit comprises at least one stimulation program for creating stimulation commands for a stimulation state. This allows individual stimulation commands to be created in the control device.

In addition or as an alternative, the computing unit comprises at least one training program for creating training commands for a training process. In addition to individual stimulation commands, individual training commands can also be created in the control device and combined with each other.

Preferably, the computing unit is connected to the data transmission device for exchanging data, whereby the creation of new stimulation commands or training commands is possible.

In particular, the computing unit is connected to the data transmission device for the exchange of sensor data from external sensors. This allows, for example, sensor data from sensors arranged on a prosthesis and GPS data to be combined with the training data.

In particular, the computing unit comprises a computational algorithm so that the stimulation commands and/or the training commands can be generated in a reproducible manner.

In particular, the calculation algorithm is a self-learning calculation algorithm, whereby historical data and current data can be easily linked together in the training device thereby autonomously improving the training programs or stimulation programs.

Preferably, the calculation algorithm processes external sensor data from external sensors of an artificial foot sole in the operating state, whereby the patient can be trained in advance, i.e. without having an artificial foot sole, with an artificial sole suitable for his circumstances.

Preferably, the control device comprises a memory unit. This allows training programs, stimulation programs, training data, stimulation data, training commands or stimulation commands to be stored in the training device.

Preferably, the housing of the training device comprises at least a second housing portion, wherein, on the second housing portion, a mounting unit for mounting the housing on a first prosthesis part of a prosthesis is provided. Using a rail system, a clip system, or bayonet-closure system as a mounting unit, the training device can be arranged on a prosthesis in a reproducible manner, in particular, on a prosthesis shaft.

Preferably, a positioning unit for positioning the training device on the first prosthesis part of a prosthesis is provided on the training device. Thus, the plurality of stimulators of the training device can be positioned exactly at their associated reinnervated nerve cell ends on the physionomy-typical nerve area portion. For example, a stop or positioning screw is used as the positioning unit.

Preferably, the housing comprises at least one further housing portion with the visualization device, whereby the user of the training device can read at least individual training data or training commands, as well as stimulation data or stimulation commands and information about the training device on the visualization device. Typically, this information on the training device includes information on the operating state of the training device and optionally include individual prompts for the user of the training device.

Preferably, a representation of the physionomy-typical nerve area portion with reinnervated nerve cell ends can be represented on the visualization device, whereby, in addition to tactile perception, visual receptors are also trained in the user's brain.

In addition or as an alternative, a representation of the physionomy-typical nerve area portion together with reinnervated nerve cell ends can be displayed on a mobile terminal device. Thus, the training device does not require a visualization device, which means that the training device has a more compact structure, thereby improving the haptics of the training device.

In addition or as an alternative, an animated representation of the physionomy-typical nerve area portion together with reinnervated nerve cell ends can be displayed together with the systematic arrangement of the plurality of stimulators. This allows the user to observe stimulations of individual stimulators at the respectively reinnervated nerve cell ends in an animated manner and associate them with their feelings/sensations, thereby improving the tactile receptors and visual receptors of the user and linking them in the user's brain.

In addition or as an alternative, the one further housing portion comprises an energy storage unit. Thus, a compact design can be produced in the housing of the training device and a battery or an accumulator can be arranged as an energy storage unit in such a way that a uniform weight distribution in the housing becomes possible. This improves the training comfort for the patient.

In particular, the energy storage unit comprises a charging coupling unit, using which the energy storage unit is inductively rechargeable by means of a separate charging unit. This allows the energy storage unit to be recharged wirelessly. For example, the charging coupling unit has at least one magnet for inductive charging of the energy storage unit.

Preferably, the control device is equipped in such a way that either a training program for training purposes or a stimulation program for stimulation by means of external sensors can be configured. This makes the training device multifunctional for various purposes.

In particular, the control device automatically detects a rehabilitation mode (reha mode) and a prosthesis mode (active mode) by means of the detection device, which is arranged on the training device. By means of this, the decision of the currently necessary program in the training device is taken for the user so he/she does not have to take the decision himself/herself.

Preferably, the detection device is an RFID unit, whereby the training device recognizes the purpose of use independently, without a plug connection having to be connected to the training device for example.

A further aspect of the invention relates to a method for operating a training device described here in the present case, in particular as a modular stimulation device comprising following the following steps:

arranging the training device at nerve area portion with nerve cell ends, in particular, with reinnervated nerve cell ends;

displaying the nerve cell ends on a visualization device of the training device or on a mobile terminal device.

With the method for operating the training device described here, it is possible to postoperatively stimulate the growth of the relevant nerve area portions or reinnervated nerve cell ends or their synapses. The user can observe the training progress or stimulation process on the visualization device or on the mobile terminal device. This stimulates not only tactile sensors in the human body of the user, but also visual receptors, which improves the overall perception and processing in the human brain—and thus the success of the training.

In addition, at least the training state or the activity state at least one from the plurality of stimulators is displayed on the visualization device or on the mobile terminal device in such a way that the user of the training device can mentally link the triggering of a certain stimulation with a certain stimulator, thereby improving the training effect.

Preferably, an adaptation of at least one stimulation command is then carried out, whereby the user has the possibility to react to individual tactile stimulations.

In addition or as an alternative, an adaptation of at least one training command is then carried out, whereby the user has the possibility to react to individual tactile stimulations and to adapt the training of individual nerve cell ends independently.

Preferably, when adapting at least one of the stimulation commands or of at least one of the training commands, one of the plurality of stimulation programs or one of the plurality of training programs is selected. For example, the user can independently control the intensity of the stimulations or adapt the training program according to his individual needs.

Another aspect of the invention concerns the use of the training device described here as a modular stimulation device for stimulating nerve cell ends, in particular, of reinnervated nerve cell ends. This improves the growth or sensitivity of the nerve cell ends on the remaining part of an extremity at a very early stage after the loss of the extremity in such a way that the acclimatization phase can be shortened for a later necessary prosthesis with the use of stimulation.

Preferably, the use of the training device described in the present case with the method for operating the training device described in the present case, whereby the visual receptors in the brain of the user are also stimulated, thereby further shorting the acclimatization phase.

A further aspect of the invention relates to a prosthesis with a first prosthesis part, wherein the first prosthesis part comprises at least one receptacle for accommodating a training device as a modular stimulation device and wherein the training device is arranged on the at least one receptacle of the first prosthesis part in a separable/removable manner.

With the prosthesis according to the invention, the user of a training device, which was used immediately after an operation to improve the growth or sensitivity of relevant nerve area portions or nerve cell ends, can then use these together with the prosthesis for use during training, but also for sensor use.

The receptacle is formed according to the invention shaped in a window-like manner and typically comprises a frame or an insert, wherein the training device is inserted within it. For example, the user, the foot of whom has been amputated, can not only use the training device as a rehabilitation module, but also as a modular stimulation device. The modular stimulation device is connected to an artificial foot sole in such a way that a rolling movement of an artificial foot along its gait line is fed back to the modular stimulation device and thus the patient can be given the feeling of walking (sensor use). This not only improves the quality of life of the user, but also minimizes the notoriously high costs for prosthesis adaptations and rehabilitation.

Favourably, the receptacle comprises at least two receptacle openings, which are suitable for the respective receptacle of at least one stimulator of a training device. This prevents a direct resting of the training device on the body part of the user so that only the stimulators can touch the body part of the user and thus train the nerve cell ends. Furthermore, the prosthesis part with the receptacle remains dimensionally stable and retains a sufficiently high level of strength. In particular, the receptacle comprises its own receptacle opening for each stimulator of a training device.

Favourably, a sealing insert, for example, made of a silicone material is arranged between the receptacle and the training device. This prevents the user's body part from being pushed outwards through the receptacle openings, which could cause skin irritations.

In particular, the first prosthesis part is a prosthesis shaft, a prosthetic stocking, or a prosthetic cuff, or a prosthesis cosmetic elements (part of the prosthesis located between the prosthesis shaft and the prosthetic foot). Depending on the amputated extremity, such as thighs, lower legs, upper arm, or forearm, the first prosthesis parts mentioned have advantages within the scope of their application. For example, the arrangement of the modular stimulation device or training device can be individually adapted to the user of the prosthesis in such a way that, depending on the need of the user or wearing comfort of the prosthetic device, the receptacle for accommodating the modular stimulation device can be positioned at different points on the prosthesis

In particular, the modular stimulation device is the training device described here.

Preferably, the first prosthesis part comprises at least one input unit for detecting the training device. Thus, the training device autonomously recognizes when it is arranged in the receptacle of the prosthesis part, which facilitates the operation of the training device for the user.

In particular, the at least one input unit is an RFID unit, whereby the arranging of the training device in the prosthesis is automatically detected.

Preferably, the first prosthesis part comprise at least one prosthesis mounting unit, whereby the training device can be easily arranged on the first prosthesis part.

Preferably, the at least one prosthesis mounting unit is complementary to the first mounting unit of the training device. Thus, a misalignment of the training device on the first prosthesis part is prevented, thereby extending the service life of the prosthesis with the training device. Typically, a rail system, a bayonet-closure system, a pinhole-hole system or another known quick-lock system are used as a prosthesis mounting unit and as a mounting unit of the training device, which facilitates the arrangement of the training system on the prosthesis for the user.

In particular, the at least one prosthesis mounting unit is arranged on the outer surface of the first prosthesis part, whereby the arrangement of the training device on the prosthesis can also be carried out in the state applied onto the user.

Preferably, a fixing unit for fixing the training device is present in the first prosthesis part. A sufficient fixation of the separable training device on the first prosthesis part is necessary in order to be able to perform different movements, such as a walking movement, a mountain-climbing movement, a bouncing movement etc., without contact being able to be lost between the plurality of stimulators of the training device and the nerve cell ends of the physionomy-typical nerve area portion.

Preferably, external sensors for the detection of pressures acting on the prosthesis are present, which are connected to the training device for receiving sensor data, wherein the external sensors are arranged in another prosthesis part of the prosthesis. As already described here, the training device has a data transmission device, which comprises, among other things, a receiving unit for receiving sensor data. Thereby, with the aid of a plurality of stimulators systematically arranged in the training device, the pressures acting on the prosthesis become transferrable to the nerve cell ends in the physionomy-typical nerve area portion in such a way that the user feels or perceives the effect of the pressure on the prosthesis.

In particular, the external sensors are arranged on an artificial foot sole. Typically, the external sensors are arranged along the gait line on the artificial foot sole so that the user of the prosthesis can perform ultrasensitive movements with the prosthesis.

In particular, the external sensors are electrically connected to a transmitting unit on the artificial foot. This allows sensor data to be sent from the artificial foot to a receiving unit so that the sensor data is evaluated externally.

Favourably, the transmitting unit is arranged in a detachable on the artificial foot or on a cosmetic element. In this case, this sending unit may comprise a retaining device and be magnetically arranged on the artificial foot or on the cosmetic element and be arranged along this in an adjustable manner. Thus, the transmitting unit can be flexibly placed and arranged on the artificial foot or on the cosmetic element according to a user's requirement, at a position that is suitable for the user of the prosthesis.

In particular, the transmitting unit comprises a charging coupling unit, using which an energy storage unit in the transmitting unit is inductively rechargeable by means of a separate charging unit. This allows the energy storage unit to be recharged wirelessly. For example, the charging coupling unit has at least one magnet for inductive charging of the energy storage unit.

In addition or as an alternative, external sensors are available for the detection of forces acting on the prosthesis, whereby the user can not only perceive pressures during a walking movement, for example, but the wearer also feels forces on the prosthesis, which take place, for example, from a thrust onto the lateral prosthesis-strip portion, or can perceive a combination of pressures and forces, whereby a realistic feeling with a prosthesis is possible.

Another aspect of the invention concerns a method for making a prosthesis comprising following the following step:
producing a receptacle for attaching a training device to a first prosthesis part of the prosthesis.

Thereby, a prosthesis can be produced, which can be individually made to the needs of the user and to which a training device can be arranged as a modular stimulation device. This allows this to be combined with a training device, which has already been used by the user for rehabilitation purposes in advance. Thus, the nerve cell ends, such as reinnervated nerve cell ends for example, can be trained by the user early on and the same training device can later be used as a modular stimulation device in a prosthesis.

Preferably, the receptacle shaped in a window-like manner so that the user can easily insert the training device into the receptacle and can simultaneously see his/her own or the physionomy-typical nerve area portion from the outside and can, if necessary, simply clean it in advance for example.

In particular, the training device is training device described herein and it is designed as a modular stimulation device. For this purpose, the training device has a systematic arrangement of the plurality of stimulators, which can be easily connected by the user to each associated nerve cell ends.

In particular, the prosthesis described here is produced using the method.

Preferably, after manufacturing the receptacle, the training device is inserted into the receptacle and fixed in the receptacle.

Another aspect of the invention relates to the use of the training device described here for stimulating nerve cell ends, in particular, of reinnervated nerve cell ends in a prosthesis described here. This minimizes the notoriously high cost for a prosthesis wearer.

Further advantages, features, and details of the invention arise from the following description, in which exemplary embodiments of the invention are described with reference to the drawings.

The reference list is also an integral part of the disclosure like the technical content of the patent claims and figures are. The figures are comprehensively described in relation to one another. Identical reference numbers denote identical components, and reference characters having different indices indicate functionally identical or similar components.

The figures show:

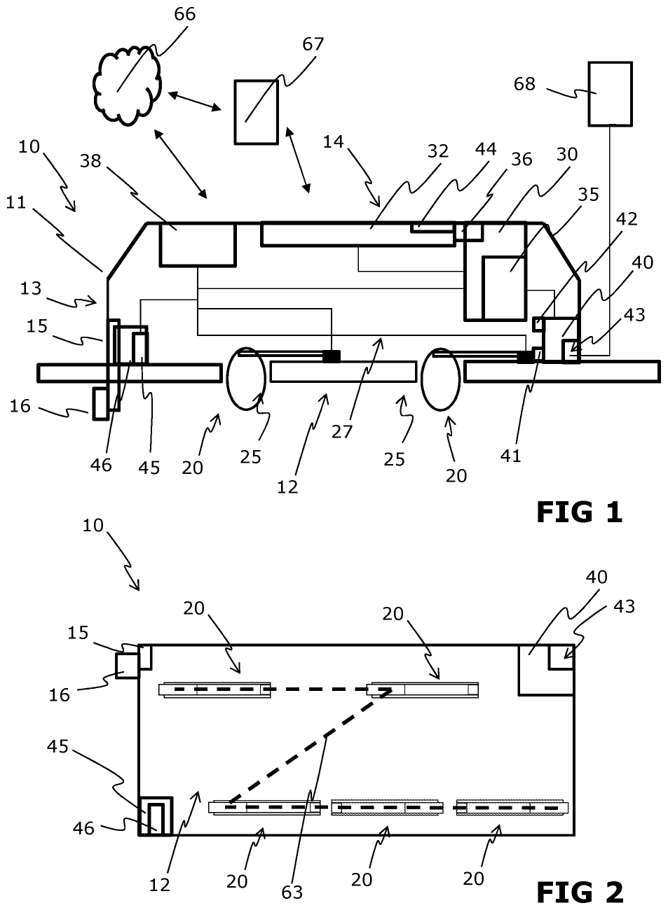

FIG. 1 a training device according to the invention in a sectional view,

FIG. 2 the training device in accordance with FIG. 1 in a top view,

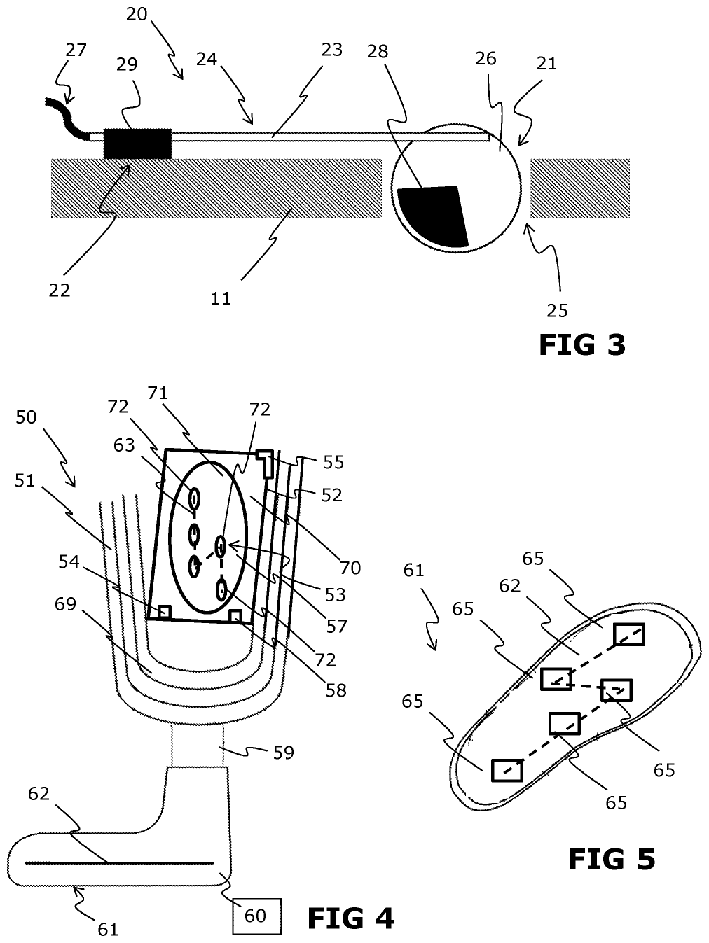

FIG. 3 one stimulator from the plurality of stimulators of the training device according to the invention in a detailed illustration,

FIG. 4 a prosthesis according to the invention without the training device in a sectional view,

FIG. 5 the artificial foot sole of the prosthesis in accordance with FIG. 4 in a top view, and

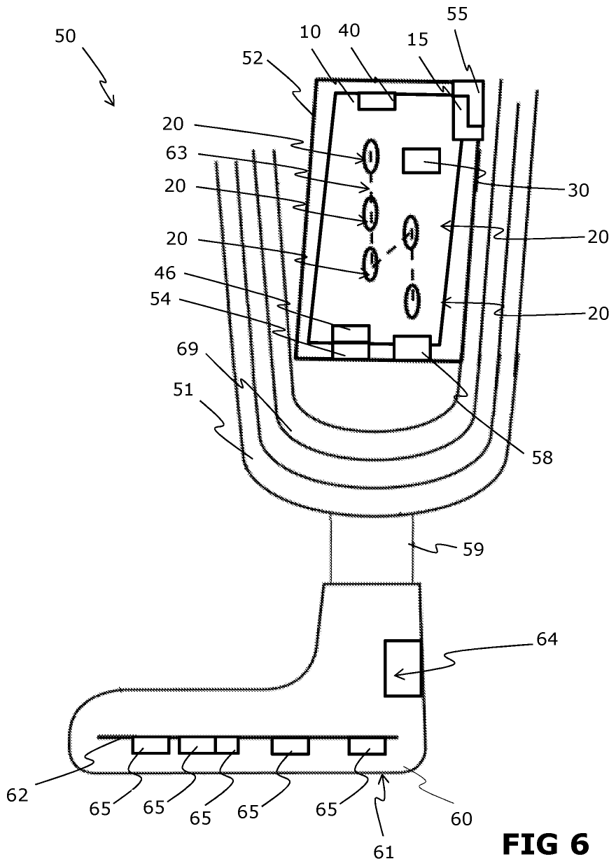

FIG. 6 the prosthesis in accordance with FIG. 4 with the training device in accordance with FIG. 1 in a perspective view.

FIG. 1 and FIG. 2 show the training device 10 according to the invention for the training stimulating of nerve cell ends. The training device 10 comprises a housing 11 with a first housing portion 12, on which plurality of stimulators 20 for stimulating physionomy-typical nerve area portions are arranged. For this purpose, the first housing portion 12 comprises a plurality of housing openings 25, in which the stimulators 20 are arranged in portions. On a second housing portion 13 of the housing 11, the training device 10 comprises a mounting unit 15 for mounting the housing 11 on a first prosthesis part of a prosthesis, as well as a positioning unit 16 for positioning the training device 10 on a first prosthesis part of a prosthesis. A control device 30 is arranged in the housing 11 for controlling the plurality of stimulators 20. The control device 30 comprises a computing unit 35, a processor, as well as a memory unit 36, which are electrically connected to each other and to the plurality of stimulators 20. In the computing unit 35, different stimulation programs are executed for creating stimulation commands for a stimulation state of the plurality of stimulators 20. Furthermore, 35 different training programs for creating training commands for a training process for the plurality of stimulators 20 are executed in the computing unit. The computing unit 20 comprises a plurality of calculation algorithms, using which the stimulation commands and/or the training commands are generated. In the memory unit 36, the plurality of calculation algorithms are stored, which are retrieved by the computing unit 35 or by the processor as required. The control device 30 is designed in such a way that at least one training program for either training purposes or a stimulation program for stimulation by means of the plurality of stimulators 20 can be configured.

In accordance with this special embodiment, the training device 10 comprises a data transmission device 40 with a transmitting unit 41 and a receiving unit 42 for wireless data exchange. The data transmission device 40 is electrically connected to the control device 30 and the computing unit 35. The data transmission device 40 further has an interface 43, with which a remote device 68, such as a computer for example, can be electrically connected and at which a training program or a stimulation program can be defined. The transmitting unit 41 or the receiving unit 42 can be connected with a cloud 66 or with a mobile terminal device 68 (smartphone, tablet, etc.) for exchanging data, for example historical data. The transmitting unit 41 is also connected to an external sensor, for example, a sensor on a prosthesis part or an external sensor, such as a temperature sensor, a humidity sensor or a GPS sensor, etc., for exchanging data. The data is transferable to the computing unit 35 and is processed there in the calculation algorithm so that the control device can transmit 30 stimulation commands and/or training commands to the plurality of stimulators 20.

The housing 11 comprises a further housing portion 14 with the visualization device 32, on which individual training data or training commands as well as stimulation data or stimulation commands and information, such as messages are displayed, being partially animated if applicable.

The visualization device 32 is electrically connected to the control device 30 and receives from the control device 30 the training data or training commands, as well as stimulation data or stimulation commands and information. The visualization device 32 comprises an input device 44, which is preferably designed as a touchscreen. In addition to a main switch (on and off switch), the input device 44 also includes a selection switch for selecting specified training programs and/or stimulation programs.

On the further housing portion, an energy storage unit 38, a battery or an accumulator is arranged, which is electrically connected to the control device 30, with the plurality of stimulators 20 and with the detection device 45. The energy storage unit 38 also supplies the visualization device 32 with energy.

On the first housing portion 12, a detection device 45, as RFID unit 46, is provided so that the control device 30 automatically recognizes the need for a rehabilitation mode (reha mode) and a prosthesis mode (active mode)—depending on what is immediately connected.

The plurality of stimulators 20 are systematically arranged on the first housing portion 12 so that the plurality of stimulators 20 in the training state can act on their respectively associated nerve cell ends. For this purpose, the systematic arrangement of the planarity of stimulators 20 displays a depiction of the gait line 63 of a human foot sole. The plurality of stimulators 20 are arranged in a lightning shape on the first housing portion 12. In the example shown in FIG. 2, five stimulators 20 are needed to display the depiction of the gait line 63 of the human foot. The systematic arrangement of the plurality of stimulators 20 is carried out in such a way that each stimulator 20 each at least coincides identically with at least one associated nerve cell end in the physionomy-typical nerve area portion.

FIG. 3 shows one of the plurality of stimulators 20 for stimulating nerve cell ends, which are arranged on the first housing portion 12. The stimulator 20 comprises a vibration generator 21 with a vibration-generator housing 26, which is connected to a spring member 23 as decoupling elements 24. The spring member 23 is a rod-shaped spring wire, which is arranged on one housing side of the vibration-generator housing 26. For this purpose, the spring member 23 is attached with one end to the vibration-generator housing 26. The spring member 23 decouples the vibrations or oscillations of the vibration generator 21 from the surrounding area. The other end of the spring member 23 is fixed with a fastening means 29 to a fastening portion 22 of the first housing portion 12 and is supplied with energy by means of the power lines 27. In this case, the stimulator 20 is arranged along the housing portion 12 in a longitudinal direction in a spatially adjustable manner. Furthermore, the stimulator 20 is arranged around the fastening means 29 in a rotatable manner, wherein the fastening means 29 can thereby be detached. For this purpose, the housing openings 25 comprise a corresponding embodiment. Alternatively, another rod-shaped spring member is arranged between the fastening portion 22 and the vibration-generator housing 26 (not shown). The vibration-generator housing 26 is introduced at least partially into the housing opening 25, so that the vibration-generator housing 26 at least partially penetrates the housing opening 25. An eccentric element 28 is arranged in the vibration-generator housing 26. The eccentric element 28 is rotatable or rotatably mounted in the vibration-generator housing 26. The vibrating of the vibration generator 21 is caused with the eccentric element 28 via the alternating direction of the centripetal force of the eccentric element 28. The vibration generator 21 has in its vibration-generator housing 26 a drive motor for rotational propulsion of the eccentric element 28 (not shown). The drive motor is connected to the energy storage unit 38. Alternatively, the vibration generator 21 has an oscillation element instead of the eccentric element, which moves along a straight guide and causes a vibration due to the mass inertia of the oscillation element (not shown).

The invention also relates to a method for operating the herein described training device 10, in particular as a modular stimulation device comprising the following steps:
arranging the training device 10 on a physionomy-typical nerve area portion 71 with nerve cell ends, in particular, with reinnervated nerve cell ends 72;
representing the nerve cell ends or the original activity site associated with them on the amputated extremity of the patient at a visualization device 32 of the training device 10 or on a mobile terminal device 67.

In addition, the plurality of stimulators 20 are displayed on the visualization device 32 or on the mobile terminal device 67 so that the user of the training device 10 can mentally link the triggering of a certain stimulation with a certain stimulator 20, thereby improving the training effect.

Subsequently, an adaptation of at least one stimulation command and/or of at least one training command on the input device 44 of the visualization device 32 or on the mobile terminal device 67 is carried out. One stimulation program from the plurality of stimulation programs or one of the plurality training programs is selected and then executed by the control device 30. The plurality of stimulators 20 are stimulated sequentially or simultaneously according to the program, so that they stimulate the nerve cell ends on the physionomy-typical nerve area portion 71.

Another aspect of the invention concerns the use of the training device described here in the present case 10 as a modular stimulation device for stimulating nerve cell ends, in particular, of reinnervated nerve cell ends.

FIG. 4 shows a prosthesis 50 with a prosthesis shaft 51 as the first prosthesis part, which is arranged at an extremity 69. The prosthesis shaft 51 comprises a receptacle 52 for accommodating a training device as a modular stimulation device. The training device can be separated from the receptacle 52 of the prosthesis shaft 51. The receptacle 52 is shaped in a window-like manner and comprises a frame, wherein the training device is inserted in it. The receptacle 52 is arranged in the region of a skin area 70 of the remaining extremity 69 and allows an access from the outside to the skin area 70. At this skin area 70, there is a physionomy-typical nerve area portion 71 with the reinnervated nerve cell ends 72 arranged there. The reinnervated nerve cell ends 72 are arranged in the physionomy-typical nerve area portion 71 in this example according to a special matrix or in a lightning-shaped manner. The reinnervated cell ends 72 lie along the depiction of a gait line 63. The prosthesis shaft 51 has an input unit 54, as an RFID unit, which acts together with the RFID unit of the training device and automatically recognizes this in the state arranged on the prosthesis 50. The prosthesis shaft 51 comprises a prosthesis mounting unit 55, whereby the training device can be arranged on the prosthesis 51. The prosthesis 50 has a prosthesis cosmetic element 59 and an artificial foot 60 with an artificial foot sole 61, which also bears a gait line 62 of the artificial foot sole 61 or rolls along it when the artificial foot performs a walking motion. The receptacle 52 comprises receptacle openings 53, which are suitable for the respective accommodation of at least one stimulator 20 of a training device. A sealing insert 57, for example, made of a silicone material, can be arranged between the receptacle 52 and the training device.

FIG. 5 shows the artificial foot sole 61 of the prosthesis 50. Five external sensors 65, which are designed as pressure sensors, are arranged on the artificial foot sole 61. The external sensors 65 are arranged in a lightning-shaped manner on the artificial sole 61 and lie along the gait line 62 of the artificial foot sole. In the embodiment shown, the shape of the depiction of the gait line 63 on the physionomy-typical nerve area portion 71 of the extremity 69 (FIG. 4) is identical to the shape of the gait line 62 of the artificial foot sole 61. However, it is possible to design the depiction of the gait line 63 independently to the gait line 62 of the artificial foot sole 61. The only decisive factor here is that the respective nerve cell ends in the physionomy-typical nerve area portion 71 are associated with the respective stimulators 20 of the training device 10.

FIG. 6 shows the prosthesis 50 described here with the training device 10 accommodated on the receptacle 52. In this process, the mounting unit 15 of the training device 10 complementary to the prosthesis mounting unit 55 on the prosthesis shaft 51. In this case, the training device 10 can be positioned with the positioning unit 16 and fastened or fixed to the prosthesis shaft 51 using the fixing unit 58 attached or fixed. The training device 10 is arranged with their systematically arranged stimulators 20 on their respectively associated nerve cell ends on the physionomy-typical nerve area portion 71 of the extremity 69 in such a way that the depiction of the gait line 63 coincides with the systematic arrangement of the plurality of stimulators 20.

On the artificial foot sole 61 of the artificial foot 60, a plurality of external sensors 65 are arranged for the detection of pressures acting on the prosthesis 50. The plurality of external sensors 65 are arranged along the gait line 62 and electrically connected to the transmitting unit 64 of the artificial foot 60. Thereby, the transmitting unit 64 is arranged in a detachable manner on the artificial foot 60, or on the cosmetic element 59. In this case, the transmitting unit 64 is magnetically arranged on the artificial foot 60 or on the cosmetic element 59 by means of a retaining device and arranged along this in an adjustable manner. The prosthesis 50 can also comprise further external sensors for detecting a force acting on the prosthesis 50. These external sensors are arranged, for example, on a prosthesis portion arranged on the side of the artificial foot 60 and transmit sensor data to the training device 10, which act by a shock on these external sensors in the prosthesis-strip portion (not shown). The transmitting unit 64 sends the sensor data from the external sensors 65 and the sensors on the prosthesis-strip portion to the receiving unit 42 of the data transmission device 40 of the training device 10. The sensor data is then forwarded to the control device 30. The transmitting unit 64 can comprise a charging coupling unit, with which an energy storage unit in the transmitting unit is inductively rechargeable by means of a separate charging unit (not shown).

As shown in FIG. 6, the prosthesis shaft 51 comprises a receptacle 52 for accommodating the training device 10. In the production of the prosthesis 50, a window-like receptacle 52 for fastening the training device 10 is produced on the prosthesis shaft 51. Alternatively, a box-shaped receptacle is produced in which the training device 10 is attached. Subsequently, the training device 10 is inserted into the window-like receptacle 52 whereby training device 10 is detected by means of its detection device 45 of the input unit 54 of the prosthesis 50 in such a way that the control device 30 automatically switches into the prosthesis mode. The transmitting unit 64 on the artificial foot 60 sends the sensor data from the external sensors 65 and the sensors on the prosthesis-strip portion to the receiving unit 42 of the data transmission device 40 of the training device 10. The sensor data is then forwarded to the control device 30 and processed there. In the computing unit 35 of the control device 30, the sensor data of the external sensors are transmitted in stimulation commands of the respectively associated stimulators 20. The respective stimulator 20 then stimulates the nerve cell end associated with it on the physionomy-typical nerve cell portion 71.

Thus, the training device 10 is usually used in a state separated from the prosthesis 50 as a training device 10 in rehabilitation mode and in the state arranged on the prosthesis 50 in prosthetic mode. Deviations from this rule can be provided, for example, if the user wishes to obtain training in the case of a prosthesis mounted on.

### REFERENCE LIST

10 training device

11 housing

12 first housing portion

13 second housing portion

14 further housing portion

15 mounting unit

16 positioning unit

20 stimulators

21 vibration generator

22 fastening portion

23 spring member

24 decoupling element

25 housing opening

26 vibration-generator housing

27 power line

28 eccentric element

29 fastening means

30 control device

32 visualization device

35 computing unit

36 memory unit

38 energy storage unit

40 data transmission device

41 transmitting unit

42 receiving unit

43 interface

44 input device

45 detection device

46 RFID unit

50 prosthesis

51 prosthesis shaft

52 receptacle

53 receptacle openings

54 input unit

55 prosthesis mounting unit

57 sealing insert

58 fixing unit

59 prosthesis cosmetic element

60 artificial foot

61 artificial foot sole

62 gait line

63 depiction of the gait line

64 transmitting unit

65 external sensors

66 cloud

66 mobile terminal device

67 remote device

68 extremity

70 skin area

71 physionomy-typical nerve area portion

72 reinnervated nerve cell end

1. A training device (10) for stimulating nerve cell ends in a training manner, comprising:
a housing (11) having at least one first housing portion (12); and
a plurality of stimulators (20) for stimulating physionomy-typical nerve area portions (71),
wherein, at least on the first housing portion (12) of the housing (11), a plurality of stimulators (20) are systematically arranged in such a way that the plurality of stimulators (20) in the training state act on respectively associated nerve cell ends.

2. The training device (10) according to claim 1, further comprising: a control device (30) for controlling the plurality of stimulators (20), which comprises at least one training program.

3. The training device (10) according to claim 2, further comprising a data transmission device (40) for transmitting data, wherein the data transmission device (40) is arranged in the control device (30) in such a way that external training data and/or stimulation data is transferred to the control device (30).

4. The training device (10) according to claim 3, wherein the data transmission device (40) is provided with external sensor data (65) of a prosthesis (50).

5. The training device (10) according to claim 2, wherein the control device (30) comprises a computing unit (35), the computing unit (35) comprises at least one stimulation program for creating stimulation commands for a stimulation state and/or at least one training program for creating training commands for a training process.

6. The training device (10) according to claim 1, wherein the housing (11) comprises at least one second housing portion (13), on the second housing portion (13), a mounting unit (15) for mounting the housing (11) on a first prosthesis part of a prosthesis (50).

7. The training device (10) according to claim 2, wherein the control device (30) is equipped in such a way that a training program for training purposes or a stimulation program for stimulation by means of external sensors (65) is configured, wherein the control device (30) automatically detects a rehabilitation mode and a prosthesis mode by means of a detection device (45).

8. A method for operating a training device (10) for stimulating nerve cell ends in a training manner, and including: (i) a housing (11) having at least one first housing portion (12); and (ii) a plurality of stimulators (20) for stimulating physionomy-typical nerve area portions (71), wherein, at least on the first housing portion (12) of the housing (11), a plurality of stimulators (20) are systematically arranged in such a way that the plurality of stimulators (20) in the training state act on respectively associated nerve cell ends, the method comprising:
arranging the training device (10) on a physionomy-typical nerve area portion (71) with nerve cell ends; and
displaying the nerve cell ends at a visualization device (32) of the training device (10) or on a mobile terminal device (68).

9. (canceled)

10. A prosthesis (50), comprising:
a first prosthesis part;
a prosthesis shaft (51) and/or a prosthesis stocking,
wherein the first prosthesis part comprises at least one receptacle (52) for accommodating a training device (10) as a modular stimulation device, wherein the training device is arranged in a separable manner on the at least one receptacle (52) of the first prosthesis part, and
wherein the training device (10) for stimulating nerve cell ends in a training manner includes: (i) a housing (11) having at least one first housing portion (12); and (ii) a plurality of stimulators (20) for stimulating physionomy-typical nerve area portions (71), wherein, at least on the first housing portion (12) of the housing (11), a plurality of stimulators (20) are systematically arranged in such a way that the plurality of stimulators (20) in the training state act on respectively associated nerve cell ends.

11. The prosthesis (50) according to claim 10, wherein the first prosthesis part comprises at least one input unit (54) for detecting the training device.

12. The prosthesis (50) according to claim 10, wherein the first prosthesis part comprises at least one prosthesis mounting unit (55), the at least one prosthesis mounting unit (55) is complementary to a mounting unit (15) of the training device (10).

13. The prosthesis (50) according to claim 10, further comprising external sensors (65) for the detection of forces acting on the prosthesis (50) and/or pressures, which are connected to the training device for receiving sensor data, wherein the external sensors (65) are arranged in a further prosthesis part of the prosthesis (50).

14. A method for producing a prosthesis (50), the prosthesis (50), comprising: a first prosthesis part; a prosthesis shaft (51) and/or a prosthesis stocking, wherein the first prosthesis part comprises at least one receptacle (52) for accommodating a training device (10) as a modular stimulation device, wherein the training device is arranged in a separable manner on the at least one receptacle (52) of the first prosthesis part, and the training device (10) for stimulating nerve cell ends in a training manner, including: (i) a housing (11) having at least one first housing portion (12); and (ii) a plurality of stimulators (20) for stimulating physionomy-typical nerve area portions (71), wherein, at least on the first housing portion (12) of the housing (11), a plurality of stimulators (20) are systematically arranged in such a way that the plurality of stimulators (20) in the training state act on respectively associated nerve cell ends, the method comprising:
producing a receptacle (52) for attaching the training device (10) on a first prosthesis part of the prosthesis (50).

15. (canceled)

16. The training device (10) according to claim 2, further comprising a visualization device (32) coupled to the control device (30) for visually display to the patient of a stimulation in a training manner from at least one training program.

17. The training device (10) according to claim 2, wherein the control device (30) is arranged in the housing (11) and is controlled using at least one of an external remote device (68).

18. The training device (10) according to claim 3, wherein the data transmission device (40) comprises an interface (43), on which a user defines training programs and/or stimulation programs, wherein the interface (43) comprises, an input device (44) for entering control data.

19. The training device (10) according to claim 5, wherein the computing unit (35) is connected to the data transmission device (40) for exchanging data, including external sensor data, and the computing unit (35) comprises a computational algorithm, which, in an operating state, processes external sensor data from external sensors (65) of an artificial foot sole (61).

20. The training device (10) according to claim 6, further comprising a positioning unit (16) for positioning the training device (10) on the first prosthesis part of a prosthesis (50).

21. The prosthesis (50) according to claim 12, wherein the at least one prosthesis mounting unit (55) is arranged on an outer surface of the first prosthesis part.

22. The prosthesis (50) according to claim 13, wherein the external sensors (65) are arranged in a further prosthesis part of the prosthesis (50), on an artificial foot sole (61) thereof.
